(19) United States
(12) Patent Application Publication
(10) Pub. No.: US 2013/0295674 A1
(43) Pub. Date: Nov. 7, 2013

(54) METHOD OF EXTRACTING EMBRYOS FROM KERNELS OF CORN

(71) Applicant: PIONEER HI-BRED INTERNATIONAL, INC., Johnston, IA (US)

(72) Inventors: Steven M. Becker, Johnston, IA (US); Laura Alice Church, Des Moines, IA (US); Samuel Roy Ellis, West Des Moines, IA (US); Amber Elizabeth Heck, Des Moines, IA (US); Casey Lynn Huck, Des Moines, IA (US); Justine Marie Olszewski, West Des Moines, IA (US)

(73) Assignee: Pioneer Hi-Bred International, Inc., Johnston, IA (US)

(21) Appl. No.: 13/874,069

(22) Filed: Apr. 30, 2013

Related U.S. Application Data
(60) Provisional application No. 61/640,837, filed on May 1, 2012.
(60) Provisional application No. 61/779,560, filed on Mar. 13, 2013.

(51) Int. Cl. C12N 5/04

(52) U.S. Cl. CPC C12N 5/04
USPC 435/412

(57) ABSTRACT

A method is provided for extracting embryos from the kernels of an ear of corn. In various embodiments, the method includes receiving an extraction solution in a vessel, receiving at least a portion of an ear of corn in the vessel, the portion of the ear of corn including a plurality of kernels having embryos and intact pericarps, sealing the vessel, subjecting the sealed vessel to a vigorous shaking motion to create an embryo mixture, and collecting the embryo mixture. In some embodiments, embryos are separated from the embryo mixture.

### CROSS REFERENCE TO RELATED APPLICATIONS

The present application claims priority from U.S. Provisional Application No. 61/640,837, filed on May 1, 2012, and U.S. Provisional Application No. 61/779,560, filed on Mar. 13, 2013, each of which is hereby incorporated herein in its entirety by reference.

### FIELD OF THE INVENTION

Various embodiments of the present invention relate generally to methods for extracting plant tissues for subsequent genetic transformation or tissue culture. More specifically, embodiments of the present invention provide a method configured to collect an embryo mixture and extract embryos from the kernels of an ear of corn.

### BACKGROUND OF THE INVENTION

The development of methods for the introduction of foreign genes into organisms has had a profound impact on fields of medicine and agriculture. While the movement of genes within plant species or between closely related plant species by traditional methods based on sexual reproduction has played an important role in crop improvement for most of this century, the pace of crop improvement by such methods has been slow and limiting due to the reliance on naturally occurring genes. Recent advances in the field of genetic engineering has led to the development of genetic transformation methods that allow the introduction of recombinant DNA, into organisms. The recombinant DNA methods which have been developed have greatly extended the sources from which genetic information can be obtained for crop improvement. Recently, new crop plant varieties, developed through recombinant DNA methods, have reached the marketplace. Genetically engineered soybeans, maize, canola and cotton are now widely utilized by North America farmers.

Rapid progress has been made in developing the tools for manipulating genetic information in plants. Plant genes are being cloned, genetic regulatory signals deciphered, and genes transferred from entirely unrelated organisms to confer new agriculturally useful traits to crop plants. Recombinant DNA methods significantly increase the gene pool available for crop improvement.

On an economic basis, maize (Zea mays L.), often referred to as corn, is the most important crop grown in the United States. The continued success of American agriculture depends, to a large extent, on the continued success of U.S. maize producers. Certainly, a key factor that has lead to and helped maintain the preeminent position of maize in U.S. agriculture is the development of improved cultivars of maize. While maize geneticists and plant breeders have improved and will continue to improve maize through classical breeding approaches, molecular biologists have recently demonstrated that genetic engineering approaches may be employed to provide maize cultivars with new traits that were not attainable through classical breeding approaches. In only a few years since their initial release, commercial cultivars that have been genetically engineered for herbicide and insect resistance, have achieved phenomenal success.

While strides have been made in the genetic transformation and tissue culture of maize, a major difficulty in producing transgenic maize plants continues to be regenerating transformed maize cells into transformed maize plants. Thus, maize scientists have focused their efforts on transforming cells that have the greatest likelihood of being regenerated into a transformed plant. Maize scientists have utilized cells derived from maize embryos that have been subjected to culture conditions that are known to promote embryogenic-tissue formation. While such cells are amenable to transformation and regeneration, the recovery of transformed maize plants from a transformation attempt has been less than desirable, Methods employing cells from embryogenic-tissue cultures are both costly and laborious because such methods involve the development and maintenance of such cultures. Methods that involve the use of embryos themselves as the source of cells for transformation or tissue culture may be more desirable, particularly if the cells from the isolated embryos can be transformed soon after isolation.

Traditional methods for extracting and/or isolating embryos from maize kernels are extremely laborious and time consuming. They often require an operator to manually remove an embryo from each kernel using a small tool, such as a microspatula or scalpel. In addition to the increased cost and process time effects associated with traditional methods, operators frequently complain of repetitive motion injuries. As a result, there is a need for an improved method of extracting embryos from maize kernels.

### BRIEF SUMMARY

The present invention provides a method for extracting embryos from the kernels of an ear of corn. In various embodiments, the method includes receiving an extraction solution in a vessel, receiving at least a portion of an ear of corn in the vessel, the portion of the ear of corn including a plurality of kernels having embryos and intact pericarps, sealing the vessel, subjecting the sealed vessel to a vigorous shaking motion to create an embryo mixture, and collecting the embryo mixture. Some embodiments may further comprise separating embryos from the embryo mixture.

In some embodiments, subjecting the vessel to a vigorous shaking motion may comprise shaking the vessel in a mechanical device. In some embodiments, receiving an extraction solution in a vessel may comprise receiving a sterile media in the vessel. In some embodiments, the extraction solution may comprise a media mixture. In some embodiments, the media mixture may comprise a mixture of deionized water, MS basal salt mixture, myo-inositol, nicotinic acid, pyridoxine.HCl, thiamine.HCl, vitamin assay casamino acids, sucrose, and glucose. In some embodiments, the extraction solution may comprise at least one of coconut water, coconut milk, calcium chloride, and cow's milk.

In some embodiments, separating embryos from the embryo mixture comprises filtering the resulting embryo mixture. In some embodiments, filtering the embryo mixture comprises pouring the embryo mixture over a series of mesh screens. In some embodiments, the series of mesh screens may comprise three mesh screens. In some embodiments, the three mesh screens may comprise a first mesh screen having a U.S. mesh size of 5, a second mesh screen having a U.S. mesh size of 10, and a third mesh screen having a U.S. mesh size of 35. In some embodiments, receiving at least a portion of an ear of corn in the vessel may comprise receiving two or more sections of the ear of corn in the vessel, the two or more sections each including a plurality of kernels having embryos and intact pericarps.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 illustrates an ear of corn and an embryo extraction vessel in accordance with an example embodiment of the present invention;

FIG. 2 illustrates an ear of corn inside an embryo extraction vessel containing sterile media in accordance with an example embodiment of the present invention;

FIG. 3 illustrates an embryo extraction vessel containing an ear of corn and sterile media loaded into a mechanical paint shaking device in accordance with an example embodiment of the present invention;

FIG. 4 illustrates a schematic diagram of an embryo extraction vessel containing a resulting embryo mixture and a series of mesh screens.

FIG. 5 illustrates a vessel that includes interior features that create a textured interior in accordance with an example embodiment of the present invention;

FIG. 6 illustrates an insert that may create a textured interior for a vessel in accordance with an example embodiment of the present invention;

FIG. 7 illustrates an embodiment of the present invention configured for recirculating a remaining extraction solution;

FIG. 8 illustrates a cross-section view of the embodiment of FIG. 7;

FIG. 9 illustrates an apparatus used to collect the embryo mixture in accordance with an example embodiment of the present invention;

FIG. 10 illustrates a cross-section view of the embodiment of FIG. 9;

FIG. 11 illustrates an apparatus used to collect the embryo mixture in accordance with an example embodiment of the present invention;

FIG. 12 illustrates a cross-section view of the embodiment of FIG. 11; and

FIG. 13 illustrates a vessel containing an ear of corn and extraction solution loaded into a mechanical device in accordance with another example embodiment of the present invention.

### DETAILED DESCRIPTION

The present invention now will be described more fully hereinafter with reference to the accompanying drawings, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout.

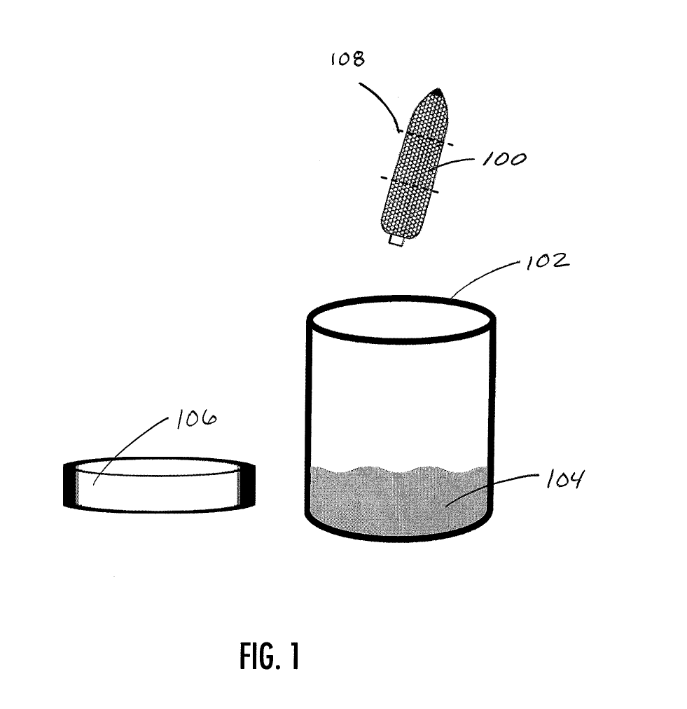

As noted above, the present invention provides a method for collecting an embryo mixture and extracting embryos from the kernels of an ear of corn. To illustrate, FIG. 1 depicts an ear of corn 100 and an embryo extraction vessel 102 in accordance with an example embodiment of the present invention. In the depicted embodiment, the embryo extraction vessel 102 includes an extraction solution 104 contained therein. A top 106 is also shown, which is configured to seal the vessel 102 shut.

In various embodiments, the process of extracting embryos from the ear of corn 100 depicted in FIG. 1 is as follows: First, an operator prepares the embryo extraction vessel 102 by ensuring that it is properly sterilized in accordance with commonly accepted laboratory procedures. It should be noted that vessel 102 of the depicted embodiment has volume of approximately ½ gallon, and has a height-to-diameter ratio similar to a standard ½ gallon paint container. In other embodiments, however, various other vessel sizes may be used. In addition, various other height-to-diameter ratios may be used. Although not intending to be bound by any particular theory, the inventors of the present invention have found that the vessel size and/or height-to-diameter ratio of the vessel may affect the forces subjected to the ear of corn during the subsequent shaking process and thus different vessel sizes and/or height-to-diameter ratios may be chosen based on a different desired extraction outcomes.

Next, the operator fills a portion of the vessel 102 with an extraction solution 104. In various embodiments, the extraction solution may or may not be a sterile solution. In the depicted embodiment, the extraction solution 104 is a media mixture. For example, in the depicted embodiment the media mixture comprises a mixture of deionized water, MS basal salt mixture, myo-inositol, nicotinic acid, pyridoxine.HCl, thiamine.HCl, vitamin assay casamino acids, 2,4-D dichlorophenoxyacetic acid, sucrose, and glucose. In some embodiments, a media mixture may include any one or any combination of these ingredients. In various other embodiments a variety of different media and media mixtures may be used. Other examples of extraction solutions may include, but need not be limited to, coconut water, coconut milk, calcium chloride, and/or cow's milk. In the depicted embodiment, a portion of the extraction solution 104 is poured into the vessel 102. In the depicted embodiment, approximately 250 ml of the extraction solution 104 is poured into the vessel 102. As such, when the sections of ear corn are added to the vessel 102, the extraction solution 104 just covers them. However, it should be noted that in other embodiments various amounts of extraction solution may be used and thus more or less than 250 ml may be used. Although not intending to be bound by any particular theory, the inventors of the present invention have also found that the amount of extraction solution 104 contained in the vessel 102 may affect the forces subjected to the ear of corn during the subsequent shaking process and thus a different volume of extraction solution may be chosen based on a different desired extraction outcome.

In some embodiments, the surface of the ear of corn 100 is then sterilized. However, in other embodiments the ear of corn may be sterilized at another time, such as in the vessel or after shaking. The ear of corn 100 is then placed into the vessel 102 containing the extraction solution 104. In various embodiments, the ear of corn 100 includes a plurality of kernels which have embryos therein. In various embodiments, the embryos may be in any stage of development. For example, in some embodiments the embryos may be immature, whereas in other embodiments the embryos may be mature. In the present invention, the pericarps of the plurality of kernels are intact (i.e., a substantial amount of the kernels have not been cut, nor the tops of the kernels been removed). In the depicted embodiment, the ear of corn 100 is broken into three sections, which are placed in the vessel 102. In other embodiments, an unbroken ear of corn 100 may be placed in the vessel 102. In still other embodiments, the ear of corn 100 may be broken into two or more sections, at least one of which is placed in the vessel 102. An example of this is depicted by dashed lines 108, which represent possible break locations that would result in three sections of the ear of corn 100. In order to preserve the sterility of the process, the ear of corn 100 may be broken into sections in a sterile environment by an operator wearing sterile gloves.

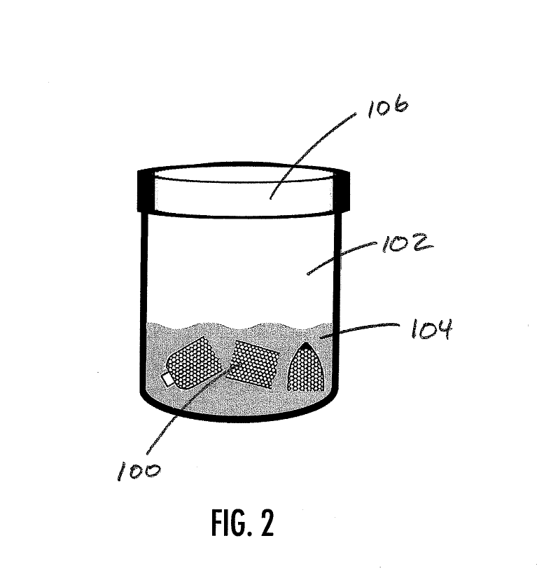

After the section(s) of the ear of corn 100, or unbroken ear of corn 100 (collectively referred to as the ear of corn 100) is placed into the vessel 102 containing the extraction solution 104, the lid 106 is placed on the vessel 102 to seal the vessel 102. In various embodiments, a variety of lid designs may be used wherein the lid is configured to seal the vessel. As above, in order to preserve the sterility of the process the lid 106 may be placed on the vessel 104 in a sterile environment by an operator wearing sterile gloves. FIG. 2 illustrates the ear of corn 100 inside the embryo extraction vessel 102 containing the extraction solution 104 in accordance with an example embodiment of the present invention.

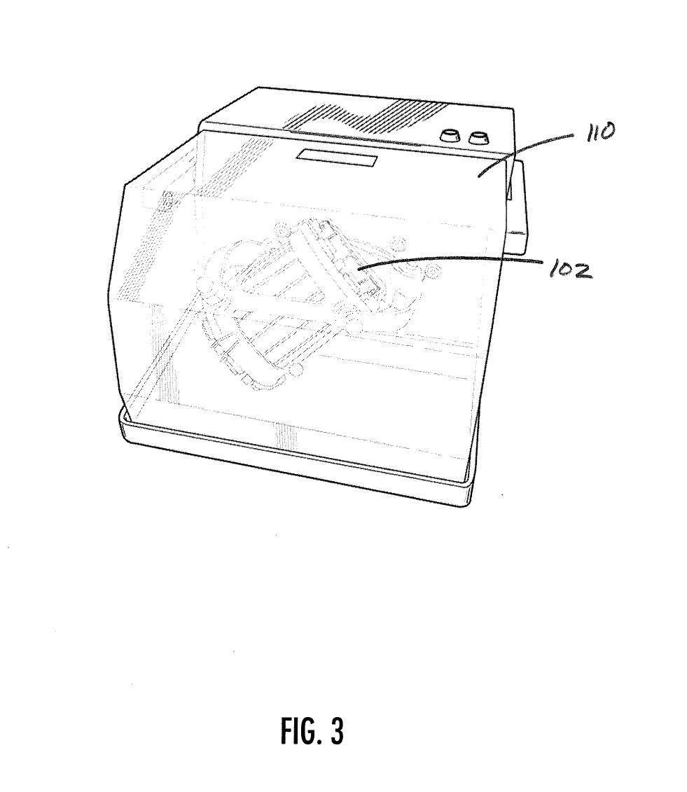

Once the vessel 102 containing the extraction solution 104 and the ear of corn 100 is sealed, it is placed in a device configured to subject the sealed vessel to a vigorous shaking motion. In the depicted embodiment, the vessel 102 is placed into a mechanical paint shaking device 110. A suitable mechanical paint shaking device is model 5G-HD Harbil® paint shaking device available from Fluid Management, Inc. of Wheeling, Illnois. In various other embodiments, however, a variety of different devices may be used. FIG. 3 illustrates the sealed embryo extraction vessel 102 containing the ear of corn and the extraction solution loaded into a mechanical paint shaking device 110.

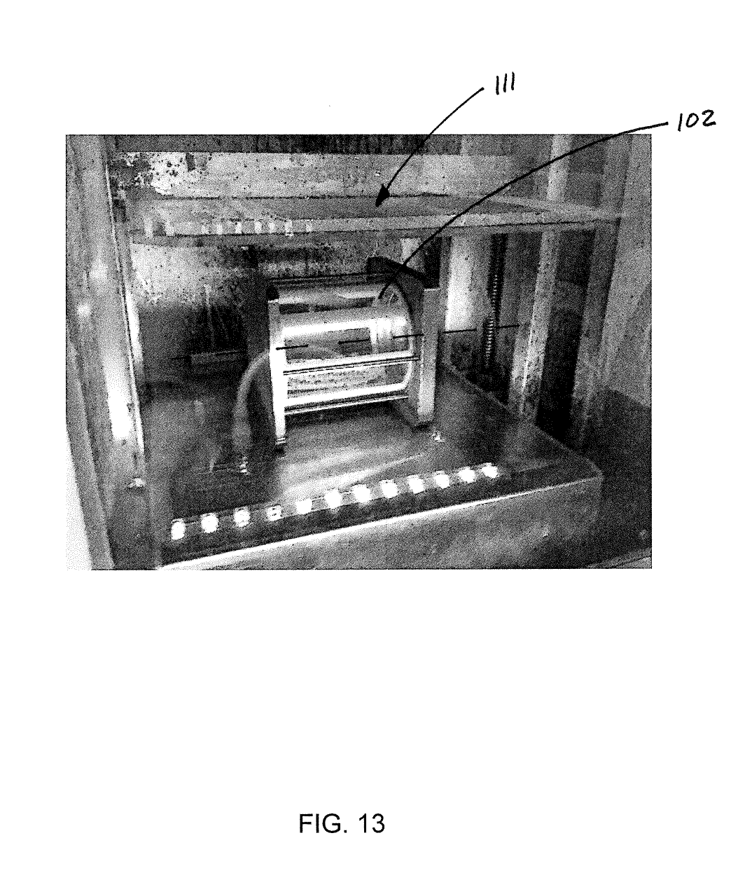

In other embodiments, the vigorous shaking motion may comprise subjecting the vessel to an orbital motion about an axis parallel to a center line of the vessel. In some embodiments, this may include maintaining the center line of the vessel in a horizontal attitude. In various embodiments, the orbital motion may be created by placing the vessel in a mechanical device configured to subject the vessel to an orbital motion about an axis parallel to a center line of the vessel. For example, FIG. 13 shows a vessel 102 containing an ear of corn and extraction solution loaded into a mechanical device 111 that is configured to subject the vessel 102 to an orbital motion about an axis parallel to a center line of the vessel 102 wherein the center line of vessel 102 is maintained in a horizontal attitude.

Once the vessel 102 is placed in the shaking device, the device is operated for a predetermined period of time. In the depicted embodiment, the device is operated for approximately 2 minutes; however in other embodiments a different operating time may be used. The shaking operation causes the ear of corn 100 to hit the inside surfaces of the vessel, which further causes the kernels to burst open. The shaking operation also causes the extraction media 102 to wash the embryos out from the kernels. Although not intending to be bound by any particular theory, the inventors of the present invention have found that the operating time of the shaking device may affect the forces subjected to the ear of corn during the subsequent shaking process and thus a different operating time may be chosen based on a different desired extraction outcome. As such, in various other embodiments different combinations of vessel sizes, vessel height-to-diameter ratios, volumes of extraction solution, and shaking device operating times may be used to vary the outcome and provide optimal results for various situations.

After the vessel 102 has been subjected to the vigorous shaking motion, such as via the mechanical paint shaking device 110 in the depicted embodiment, the vessel contains a resulting embryo mixture 112, which may be collected. In some embodiments, further processing of the resulting embryo mixture 112 may occur.

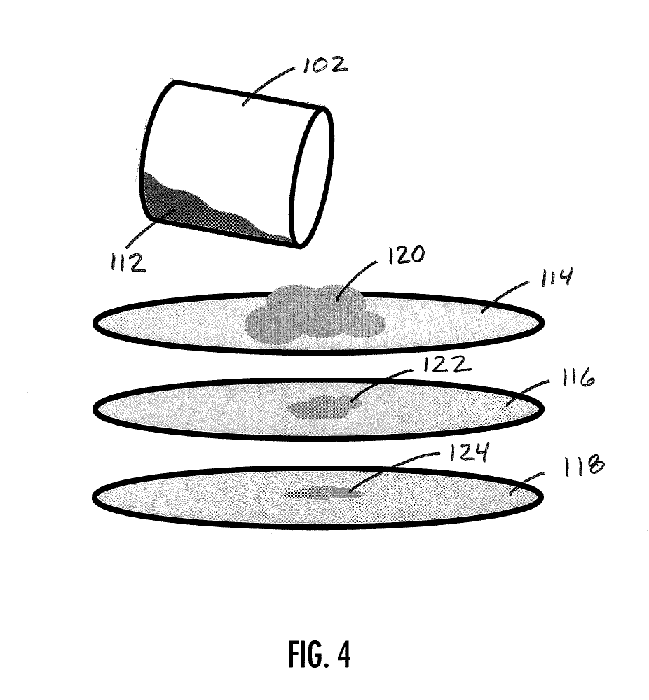

In some embodiments, embryos may be separated from the embryo mixture 112. Referring to FIG. 4, embryos are separated from the embryo mixture 112 by filtering the embryo mixture 112. FIG. 4 illustrates a series of mesh screens. In the depicted embodiment, the series of mesh screens comprises three mesh screens: mesh screen 114, mesh screen 116, and mesh screen 118, each of which has an increased degree of filtration. In such a manner, larger non-embryo material 120 (such as, for example, the remaining ear or ear sections) are filtered out at screen 114, smaller non-embryo material 122 (such as, for example, remaining kernel portions) are filtered out at screen 116, and the embryos 124 are filtered out at screen 118. Although in various embodiments, any number of screens (including as few as one) and mesh sizes may be used, in the depicted embodiment, screen 114 is 5 mesh screen (i.e., U.S. mesh size of 5 having 4000 micron openings), screen 116 is a 10 mesh screen (i.e., U.S. mesh size of 10 having 2000 micron openings), and screen 118 is a 35 mesh screen (i.e., U.S. mesh size of 35 having 500 micron openings). In the depicted embodiment, once the embryo mixture 112 passes through the series of screens, the operator may collect the embryos 124 from screen 118, such as by using a small sterilized tool. From here, in some embodiments the collected embryos 124 may be plated on cultivation media for subsequent genetic transformation. In other embodiments, the embryos or may be used for various other applications.

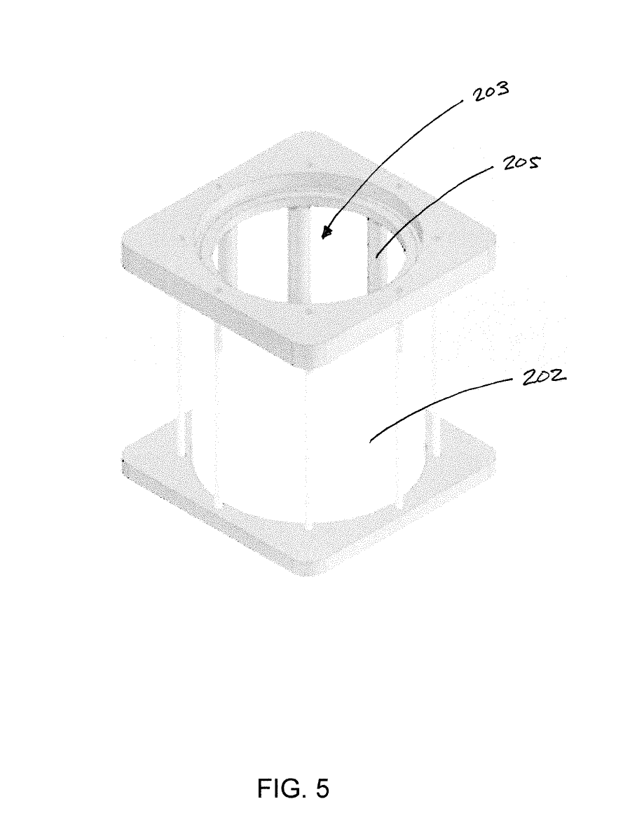

In some embodiments the vessel may include a modified interior in order to facilitate extraction of the embryos. For example, FIG. 5 illustrates an embodiment of a vessel 202 that includes interior features 203 that create a textured interior. Although in various embodiments features creating a textured interior may have a variety of configurations, in the depicted embodiment the interior features 203 comprise a plurality of bumps 205 that are spaced (in the depicted embodiment equally) along an inside wall of the vessel 202. Each bump 205 of the depicted embodiment has a partial cylindrical shape proximate an inside wall of the vessel 202, and the plurality of bumps 205 are arranged parallel to a center line of the vessel 202 (center line shown in FIG. 13).

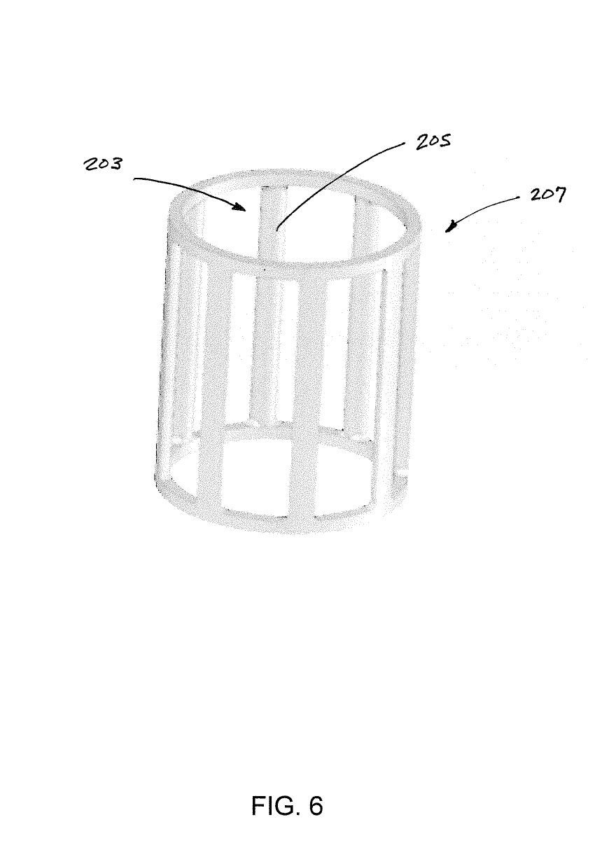

In various embodiments, the interior features may be integrated with vessel 202 or may be part of an insert that may be placed inside the vessel 202. FIG. 6 illustrates an insert 207 that comprises a plurality of bumps 205 that are spaced along a diameter of the insert 207 and that are configured, when the insert 207 in placed inside the vessel 202, to be proximate an inside wall of the vessel 202. In such a manner the insert 207 may create a textured interior for the vessel 202.

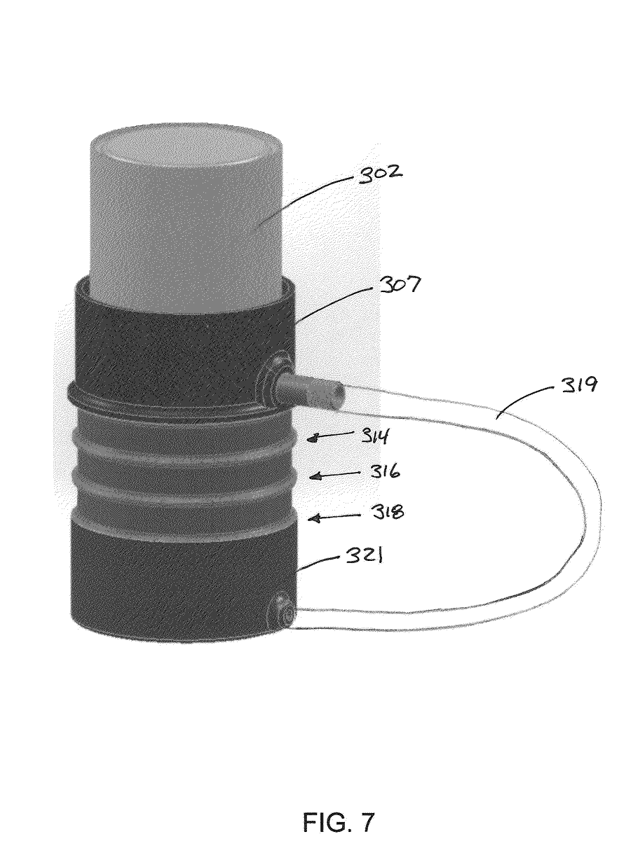

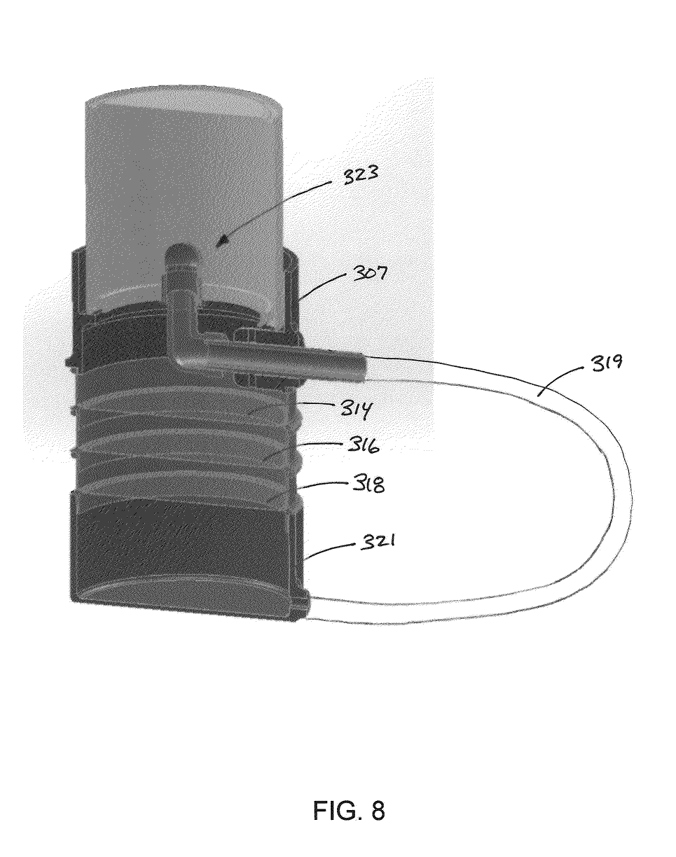

In some embodiments, embryos may be separated from an embryo mixture by recirculation, which may comprise selectively recirculating a remaining extraction solution. FIGS. 7 and 8 illustrate an embodiment of the present invention in which a vessel 302 is shown in an inverted orientation and installed into a recirculation base 307. In various embodiments, the vessel 302 may be placed in the recirculation base 307 after an embryo mixture has been emptied onto a first mesh screen 314, of a series of mesh screens 314, 316, 318. As with other embodiments of the present invention, in some embodiments the series of mesh screens may comprise mesh screens of different degrees of filtration. In the depicted embodiment, the remaining extraction solution is recirculated via a recirculation tube 319 which connects a collecting tank 321, positioned below the series of screens, to a sprayer, such as, for example, a clean-in-place ball sprayer 323. As such, after the original embryo mixture is emptied onto the first mesh screen 314, the vessel 302 may be positioned into the recirculation base 307 such that the vessel surrounds the clean-in-place ball sprayer 323 and, in conjunction with the recirculation base 307, substantially encloses the clean-in-place ball sprayer 323. In such a manner, extraction solution remaining after the embryo mixture has been filtered may be redirected to the clean-in-place ball sprayer 323 and sprayed against the interior of the vessel 302, thus facilitating the removal of any additional material that may remain along the interior of the vessel 302 by rinsing the inside of the vessel 302. As such, a subsequent embryo mixture may then pass through the series of mesh screens 314, 316, 318. In other embodiments of the present invention, the inside of the vessel 302 may be rinsed by recirculating remaining extraction solution to a hand held spray wand that may be used to direct a subsequent embryo mixture through the series of mesh screens.

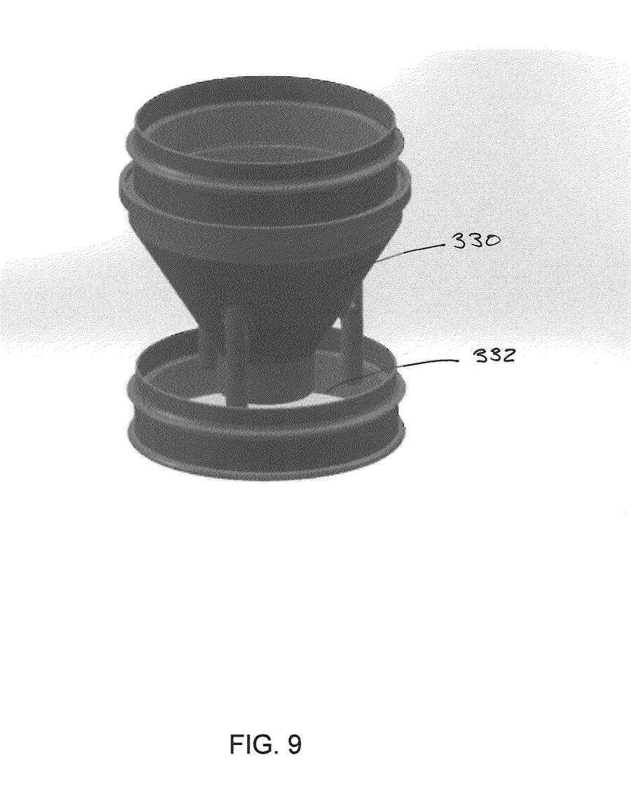

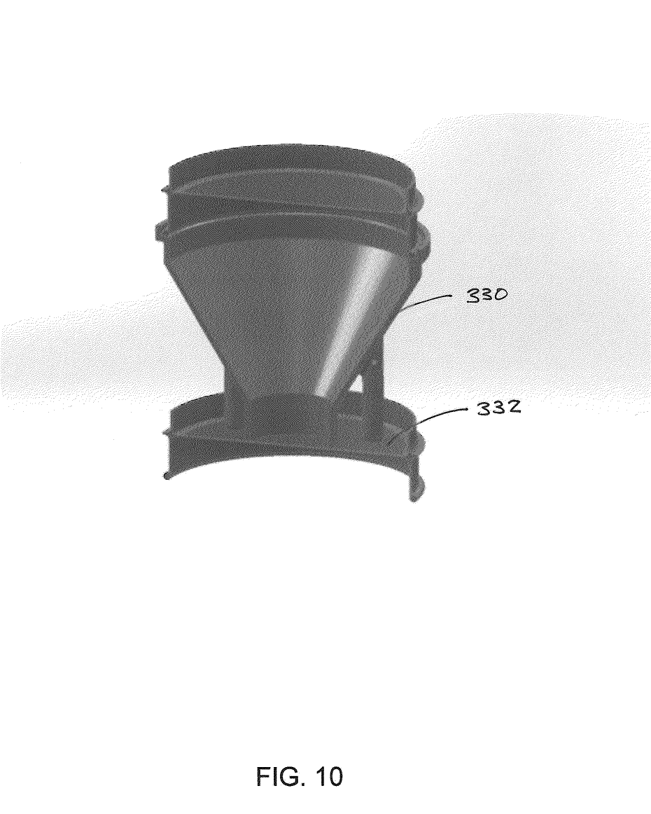

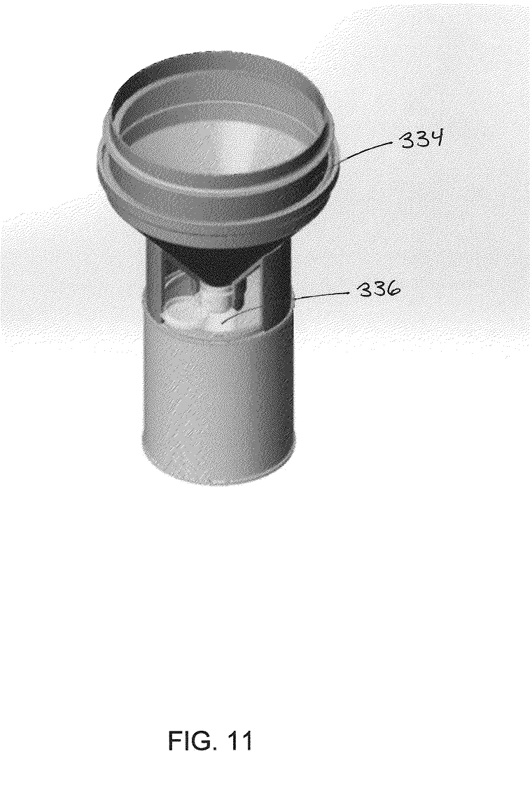

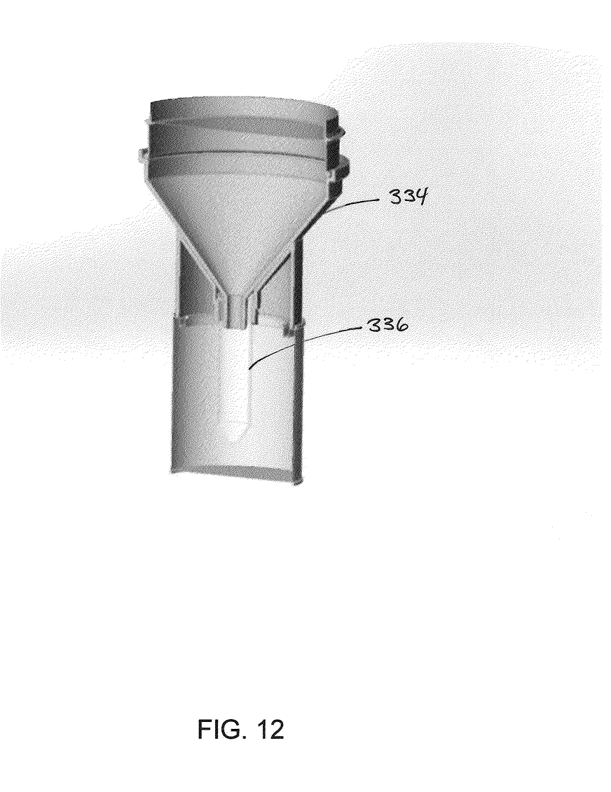

In addition to the collection methods described above, in other embodiments the embryo mixture may be collected in various other ways. For example, FIGS. 9-12 illustrate apparatuses used to collect the embryo mixture in accordance with other example embodiments of the present invention. In the embodiment depicted in FIGS. 9 and 10, a funnel 330 is used in conjunction with one or more mesh screens 332 to concentrate the embryo mixture on top of the mesh screen 332. In the embodiment depicted in FIGS. 11 and 12, a funnel 334 is used to concentrate an embryo mixture into a vial 336, which, in some embodiments, may be a graduated vial.

In such a manner, the present invention provides an improved process for extracting embryos from the kernels of an ear of corn. In various embodiments, the present invention increases the amount of embryos that may be extracted during a given time period, while reducing the amount of manual labor required. Similarly, the present invention decreases required repetitive motions commonly associated with operator injuries.

Many modifications and other embodiments of the invention set forth herein will come to mind to one skilled in the art to which these invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A method for extracting embryos from the kernels of an ear of corn, said method comprising:
receiving an extraction solution in a vessel;
receiving at least a portion of an ear of corn in the vessel, the portion of the ear of corn including a plurality of kernels having embryos and intact pericarps;
sealing the vessel;
subjecting the sealed vessel to a vigorous shaking motion to create an embryo mixture; and
collecting the embryo mixture.

2. The method of claim 1, further comprising separating embryos from the embryo mixture.

3. The method of claim 1, wherein subjecting the vessel to a vigorous shaking motion comprises shaking the vessel in a mechanical device.

4. The method of claim 1, wherein receiving an extraction solution in a vessel comprises receiving a sterile media in the vessel.

5. The method of claim 1, wherein the extraction solution comprises a media mixture.

6. The method of claim 5, wherein the media mixture comprises a mixture of deionized water, MS basal salt mixture, myo-inositol, nicotinic acid, pyridoxine.HCl, thiamine.HCl, vitamin assay casamino acids, sucrose, and glucose.

7. The method of claim 1, wherein the extraction solution comprises at least one of coconut water, coconut milk, calcium chloride, and cow's milk.

8. The method of claim 2, wherein separating embryos from the embryo mixture comprises filtering the embryo mixture.

9. The method of claim 8, wherein filtering the embryo mixture comprises pouring the embryo mixture over a series of mesh screens.

10. The method of claim 9, wherein the series of mesh screens comprises at least three mesh screens.

11. The method of claim 10, wherein the series of mesh screens comprises six mesh screens: a first mesh screen having a U.S. mesh size of 12, a second mesh screen having a U.S. mesh size of 14, a third mesh screen having a U.S. mesh size of 16, a fourth mesh screen having a U.S. mesh size of 18, a fifth mesh screen having a U.S. mesh size of 20, and a sixth mesh screen having a U.S. mesh size of 35.

12. The method of claim 1, wherein receiving at least a portion of an ear of corn in the vessel, comprises receiving a section of the ear of corn in the vessel, the section including a plurality of kernels having embryos and intact pericarps.

13. The method of claim 1, wherein subjecting the sealed vessel to a vigorous motion comprises subjecting the vessel to an orbital motion about an axis parallel to a center line of the vessel.

14. The method of claim 13, further comprising maintaining the vessel's center line in a horizontal attitude.

15. The method of claim 1, wherein receiving an extraction solution in a vessel comprises receiving an extraction solution in a vessel that includes a textured interior.

16. The method of claim 15, wherein the textured interior comprises a plurality of bumps, each bump comprising a partial cylindrical shape proximate an inside wall of the vessel and arranged parallel to a center line of the vessel.

17. The method of claim 2, wherein separating embryos from the embryo mixture comprises recirculating a remaining extraction solution.

18. The method of claim 17, wherein recirculating the remaining extraction solution comprises selectively recirculating the remaining extraction solution through a clean-in-place ball sprayer.

19. The method of claim 18, wherein recirculating the remaining extraction solution through a clean-in-place ball sprayer comprises rinsing an inside of the vessel.

20. The method of claim 19, further comprising passing a subsequent embryo mixture through a series of mesh screens.

21. The method of claim 20, wherein the series of mesh screens comprises at least three mesh screens.

22. The method of claim 21, wherein the series of mesh screens comprises six mesh screens: a first mesh screen having a U.S. mesh size of 12, a second mesh screen having a U.S. mesh size of 14, a third mesh screen having a U.S. mesh size of 16, a fourth mesh screen having a U.S. mesh size of 18, a fifth mesh screen having a U.S. mesh size of 20, and a sixth mesh screen having a U.S. mesh size of 35.

23. The method of claim 17, wherein recirculating the remaining extraction solution comprises selectively recirculating the extraction fluid through a hand held spray wand.

24. The method of claim 1, wherein the vessel includes a vertically oriented center line.

25. The method of claim 23, wherein recirculating the extraction fluid through a hand held spray wand comprises spraying a remaining extraction solution through a series of mesh screens.

26. The method of claim 25, wherein the series of mesh screens comprises at least three mesh screens.

27. The method of claim 26, wherein the series of mesh screens comprises six mesh screens: a first mesh screen having a U.S. mesh size of 12, a second mesh screen having a U.S. mesh size of 14, a third mesh screen having a U.S. mesh size of 16, a fourth mesh screen having a U.S. mesh size of 18, a fifth mesh screen having a U.S. mesh size of 20, and a sixth mesh screen having a U.S. mesh size of 35.

28. The method of claim 2, wherein separating embryos from the embryo mixture comprises using a funnel to concentrate the embryo mixture.

29. The method of claim 28, further comprising using a screen to concentrate the embryo mixture.

30. The method of claim 28, further comprising collecting the embryos in a vial.
